(19) United States
(12) Patent Application Publication
(10) Pub. No.: US 2015/0259712 A1
(43) Pub. Date: Sep. 17, 2015

(54) FATTY ACIDS WITH MG TRANSPORTER AND MG

(71) Applicant: William Marsh Rice University, Houston, TX (US)

(72) Inventors: Ka-Yiu SAN, Houston, TX (US); SongI HAN, Houston, TX (US); Wei LI, Houston, TX (US); Mai LI, Houston, TX (US); Zhilin LI, Houston, TX (US)

(21) Appl. No.: 14/642,260

(22) Filed: Mar. 9, 2015

Related U.S. Application Data
(60) Provisional application No. 61/951,010, filed on Mar. 11, 2014.

(51) Int. Cl. C12P 7/64; C12N 9/16; C07K 14/245; C07K 14/255

(52) U.S. Cl. CPC C12P 7/6409; C07K 14/255; C12N 9/16; C07K 14/245; C12Y 301/02014

(57) ABSTRACT

Methods of improving fatty acid production in bacteria, yeast, algae, and various other microbes are presented, by either supplementing the medium with millimolar amounts of magnesium, by overexpressing a magnesium transporter, or both.

### PRIOR RELATED APPLICATIONS

This application claims priority to U.S. Ser. No. 61/951,010, filed Mar. 11, 2014, and incorporated by reference herein in its entirety for all purposes.

### FEDERALLY SPONSORED RESEARCH STATEMENT

This invention was made with government support under Grant No: EEC-0813570 awarded by the NSF. The government has certain rights in the invention.

### FIELD OF THE DISCLOSURE

The disclosure generally relates to methods of improving fatty acid production in bacteria, yeast, algae and various other microbes.

### BACKGROUND OF THE DISCLOSURE

Increasing energy costs and environmental concerns have emphasized the need to produce sustainable renewable fuels and chemicals. Fatty acids are composed of long alkyl chains and represent nature's “petroleum,” being a primary metabolite used by cells for both chemical and energy storage functions. Today, these energy-rich molecules are isolated from plant and animal oils for a diverse set of products ranging from fuels to oleochemicals.

Whereas microbial fermentation processes for producing ethanol and related alcohol biofuels are well established, biodiesel (methylesters of fatty acids) is the major long chain product produced biologically, and it is almost exclusively derived from plant oils today. However, slow cycle times for engineering oil seed metabolism and the excessive accumulation of glycerol as a byproduct in biodiesel generation are two major drawbacks of deriving biodiesel from plants.

Although most bacteria do produce fatty acids as cell envelope precursors, the biosynthesis of fatty acids is tightly regulated at multiple levels and large quantities are not made, especially since free short chain fatty acids are toxic. Thus, the production of fatty acids from bacteria has not yet reached the point where it is cost effective.

The ability to produce free fatty acid at high yields and high rates by the metabolically engineered strains provides an efficient framework to produce a large class of other derived products (chemicals and biofuels) either biologically or chemically. For example, as shown in FIG. 1, by introducing additional appropriate pathways, the fatty acids can be converted to chemicals such as hydrocarbons, fatty alcohols, hydroxyl fatty acids, dicarboxylic acids, etc.

Likewise, the omega-end of the molecules can be modified by changing the starting precursors in the initial step of the fatty acid biosynthesis pathway (marked by white block arrows). Furthermore, the chain length of these molecules can be changed by using appropriate acyl-ACP thioesterases specific to a particular chain length, such as C8, C12 or C14. In addition, various molecules can also be tapped out at different points during the fatty acid elongation cycle.

US20140093921 discloses hybrid ACP thioesterases, which can be combined with deletions, such as in native fadD and sucC. It also teaches acidifying the medium to increase production of fatty acids.

Although progress towards bioproduction of fats in microbial systems has been made, there is always need in the art for a biological system of producing fatty acids that is even more efficient and cost effective than heretofore realized.

### SUMMARY OF THE INVENTION

The present invention established a simple method to increase the production of free fatty acid (FFA) in microbes by adding at least one overexpressed magnesium transporter gene to the microbe and/or by supplementing the culture medium with magnesium. These genes and/or culture methods can be combined with any microbe engineered to produce more free fatty acid than the wild type microbe, and preferably is combined with a microbe that also contains one or more overexpressed ACP thioesterase genes, per the patents and literature cited herein.

In additional preferred embodiments, the microbes are also combined with various other modifications to further improve FFA synthesis, FFA export, reduce competition for carbon and the like, including without limitation, one or more of the following:
an improved fatty acid export system to further improve viability, e.g FadL
deletion of fadR
deletion of marR, the repressor of marA expression, leading to the constitutive expression of tolC, acrA and acrB, which builds an efflux system for organic solvents
Overexpression fabL
a combination of both deletions (fadR and marR) and/or overexpression fabL
deletion of beta oxidation genes, such as one or more of fadD or fadE or fadL
deletion of genes in fermentative pathways, such as those for acetate, lactate, formate or ethanol production
overexpression of the acetyl-CoA carboxylase (ACC)
fabF overexpression
overexpression of genes for conversion of FFA to FFAE or TAG
A reverse beta oxidation pathway (e.g., as described by Gonzales)
Additional modifications are described in Janβen 2014.
a fabH-independent platform, as described in 62/120,232, filed Feb. 24, 2015 and incorporated by reference herein in its entirety for all purposes.
an NAD-dependent FabG as described in PCT/US14/59319, filed Oct. 6, 2014 and 61/889,166, filed Oct. 10, 2013, each incorporated by reference herein in its entirety for all purposes.

The magnesium transporter genes can be any of the hundreds of known transporter genes, but preferably is one whose protein has increased specificity for magnesium ions over other ions such as manganese.

The magnesium in the culture medium can be in any suitable form, e.g. magnesium carbonate, magnesium acetate, magnesium sulphate, magnesium chloride and the like, but organic forms of magnesium are preferred, such as magnesium glutamate.

The commonly used Mg concentration is 10 to 20 mM. The invention can also use more than this level, and we have surprisingly discovered that it increases fatty acid production level enormously (even without the added transporter gene). Amounts of magnesium can vary, and a simple titration experiment will indicate what level of magnesium addition is optimal for fatty acid production with and without the added transporter gene.

Exemplary levels of Mg⁺⁺ are >20 mM, >30 mM, 50 mM, >75 mM, >100 mM, >150 mM, >200 mM, >300 mM, >400 mM, and >500 mM. We have not yet titrated a maximal level, but it is anticipated that less magnesium will be required when combined with a microbe that overexpresses a magnesium transporter gene, since that microbe will more readily take up available magnesium and thus be more efficient at recovering magnesium from the medium than the cells without the added transporter.

Acyl-acyl carrier protein (ACP) thioesterase (aka “TE” herein) is an enzyme that terminates the intraplastidial fatty acid synthesis in plants by hydrolyzing the acyl-ACP intermediates and releasing free fatty acids to be incorporated into glycerolipids, as seen in FIG. 2. These enzymes are classified in two families, FatA and FatB, which differ in amino acid sequence and substrate specificity. Generally speaking, the N terminal (aa 1-98) of any acyl-ACP thioesterases controls the substrate specificity of the enzyme, and it is known how to change substrate specificity by swapping amino terminal domains.

Many acyl-ACP thioesterase proteins are known and can be added to bacteria for use in the invention (e.g., CAA52070, YP_{—}003274948, ACY23055, AAB71729, BAB33929, to provide the accession numbers for a few of the thousands of such proteins available). Such genes can be added by plasmid or other vector, or can be cloned directly into the genome. Alternatively, periplasmic expression of TesA (native E. coli thioesterase) in E. coli can be used, since this uncouples inhibition and allows the production of increased FFA.

Other acyl-ACP thioesterases include Umbellularia californica (AAC49001), Cinnamomum camphora (Q39473), Umbellularia californica fatty acyl-ACP thioesterase (Q41635), Myristica fragrans (AAB71729), Myristica fragrans (AAB71730), Elaeis guineensis (ABD83939), Elaeis guineensis (AAD42220), Populus tomentosa (ABC47311), Arabidopsis thaliana (NP_{—}172327), Arabidopsis thaliana (CAA85387), Arabidopsis thaliana (CAA85388), Gossypium hirsutum (Q9SQI3), Cuphea lanceolata (CAA54060), Cuphea hookeriana (AAC72882), Cuphea calophylla subsp. mesostemon (ABB71581), Cuphea lanceolata (CAC19933), Elaeis guineensis (AAL15645), Cuphea hookeriana (Q39513), Gossypium hirsutum (AAD01982), Vitis vinifera (CAN81819), Garcinia mangostana (AAB51525), Brassica juncea (ABI18986), Madhuca longifolia (AAX51637), Brassica napus (ABH11710), Oryza sativa (indica cultivar-group) (EAY86877), Oryza sativa (japonica cultivar-group) (NP-001068400), Oryza sativa (indica cultivar-group) (EAY99617), and Cuphea hookeriana (AAC49269).

In some embodiments, at least one acyl-ACP thioesterase gene is from a plant, for example overexpressed TE from Ricinus communis (XP_{—}002515564.1), Jatropha curcas (ABU96744.1), Diploknema butyracea (AAX51636.1), Cuphea palustris (AAC49180.1), or Gossypium hirsutum (AAF02215.1 or AF076535.1), or an overexpressed hybrid TE comprising different thioesterase domains operably fused together (see WO2011116279, all sequences expressly incorporated by reference herein). Preferably, the hybrid thioesterase includes an amino terminal region (˜aa 1-98 controls substrate specificity) of the acyl-ACP thioesterase from Ricinus communis or a 70, 80, 90 or 95% homolog thereto, or any TE with the desired substrate specificity, operably coupled to the remaining portion of the thioesterase from another species. In such manner, enzyme specificity can be tailored for the use in question.

A great number of TE proteins were characterized by Jing, and some of his results are reproduced in FIG. 3A-B.

Thus it can be seen that hundreds of such TE proteins have been used in the art, and are readily available for overexpression uses in the claimed microbes.

Magnesium transporters are not limited to E. coli MgtA (NP_{—}313246), but include Shigella dysenteriae 1617 (YP_{—}008853027), Shigella sonnei 53G (YP_{—}005459577), Shigella boydii CDC 3083-94 (YP_{—}001882973), Shigella flexneri 2002017 (YP_{—}005729836), Salmonella enterica subsp. enterica serovar Heidelberg str. 41578 (YP_{—}008245738), Klebsiella oxytoca E718 (YP_{—}006496302), Raoultella ornithinolytica B6 (YP_{—}007875494), Citrobacter koseri ATCC BAA-895 (YP_{—}001455082), Citrobacter rodentium ICC168 (YP_{—}003366794), Klebsiella pneumoniae JM45 (YP_{—}008427516), Dickeya dadantii 3937 (YP_{—}003881914), Enterobacteriaceae bacterium strain FGI 57 (YP_{—}007342088), Salmonella bongori NCTC 12419 (YP_{—}004732663), Enterobacter asburiae LF7a (YP_{—}004827006), Enterobacter cloacae subsp. cloacae ENHKU01 (YP_{—}006577020), Enterobacter cloacae EcWSU1 (YP_{—}004950323), Klebsiella oxytoca E718 (YP_{—}006496302), Raoultella ornithinolytica B6 (YP_{—}007875494), Citrobacter koseri ATCC BAA-895(YP_{—}001455082), Klebsiella variicola At-22 (YP_{—}003441505); Streptococcus pneumonia (POA3M5); Neisseria gonorrhoeae (O85665); Thermotoga maritime (Q9WZY8); ALR2 from Saccharomyces cerevisiae (P43553).

Many additional transporters are available in GenBank and can be located either by homology search, or by using a database entry point, such as UNiProt, Brenda, and the like, which collects and annotates all available protein and DNA sequences. Indeed, hundreds of genomes have been completely sequenced, and every single one will contain at least one Mg transporter gene, since Mg is an essential element for all living cells. Thus, a Mg transporter gene is available in at least the following species:

Aeropyrum pernix, Agrobacterium tumefaciens, Anabaena, Anopheles gambiae, Apis mellifera, Aquifex aeolicus, Arabidopsis thaliana, Archaeoglobus fulgidus, Ashbya gossypii, Bacillus anthracis, Bacillus cereus, Bacillus halodurans, Bacillus licheniformis, Bacillus subtilis, Bacteroides fragilis, Bacteroides thetaiotaomicron, Bartonella henselae, Bartonella Quintana, Bdellovibrio bacteriovorus, Bifidobacterium longum, Blochmannia floridanus, Bordetella bronchiseptica, Bordetella parapertussis, Bordetella pertussis, Borrelia burgdorferi, Bradyrhizobium japonicum, Brucella melitensis, Brucella suis, Buchnera aphidicola, Burkholderia mallei, Burkholderia pseudomallei, Caenorhabditis briggsae, Caenorhabditis elegans, Campylobacter jejuni, Candida glabrata, Canis familiaris, Caulobacter crescentus, Chlamydia muridarum, Chlamydia trachomatis, Chlamydophila caviae, Chlamydophila pneumonia, Chlorobium tepidum, Chromobacterium violaceum, Ciona intestinalis, Clostridium acetobutylicum, Clostridium perfringens, Clostridium tetani, Corynebacterium diphtheria, Corynebacterium efficiens, Coxiella burnetii, Cryptosporidium hominis, Cryptosporidium parvum, Cyanidioschyzon merolae, Debaryomyces hansenii, Deinococcus radiodurans, Desulfotalea psychrophila, Desulfovibrio vulgaris, Drosophila melanogaster, Encephalitozoon cuniculi, Enterococcus faecalis, Erwinia carotovora, Escherichia coli, Fusobacterium nucleatum, Gallus gallus, Geobacter sulfurreducens, Gloeobacter violaceus, Guillardia theta, Haemophilus ducreyi, Haemophilus influenza, Halobacterium, Helicobacter hepaticus, Helicobacter pylori, Homo sapiens, Kluyveromyces waltii, Lactobacillus johnsonii, Lactobacillus plantarum, Legionella pneumophila, Leifsonia xyli, Lactococcus lactis, Leptospira interrogans, Listeria innocua, Listeria monocytogenes, Magnaporthe grisea, Mannheimia succiniciproducens, Mesoplasma forum, Mesorhizobium loti, Methanobacterium thermoautotrophicum, Methanococcoides burtonii, Methanococcus jannaschii, Methanococcus maripaludis, Methanogenium frigidum, Methanopyrus kandleri, Methanosarcina acetivorans, Methanosarcina mazei, Methylococcus capsulatus, Mus musculus, Mycobacterium bovis, Mycobacterium leprae, Mycobacterium paratuberculosis, Mycobacterium tuberculosis, Mycoplasma gallisepticum, Mycoplasma genitalium, Mycoplasma mycoides, Mycoplasma penetrans, Mycoplasma pneumonia, Mycoplasma pulmonis, Mycoplasm mobile, Nanoarchaeum equitans, Neisseria meningitides, Neurospora crassa, Nitrosomonas europaea, Nocardia farcinica, Oceanobacillus iheyensis, Onions yellows phytoplasma, Oryza sativa, Pan troglodytes, Pasteurella multocida, Phanerochaete chrysosporium, Photorhabdus luminescens, Picrophilus torridus, Plasmodium falciparum, Plasmodium yoelii yoelii, Populus trichocarpa, Porphyromonas gingivalis Prochlorococcus marinus, Propionibacterium acnes, Protochlamydia amoebophila, Pseudomonas aeruginosa, Pseudomonas putida, Pseudomonas syringae, Pyrobaculum aerophilum, Pyrococcus abyssi, Pyrococcus furiosus, Pyrococcus horikoshii, Pyrolobus fumarii, Ralstonia solanacearum, Rattus norvegicus, Rhodopirellula baltica, Rhodopseudomonas palustris, Rickettsia conorii, Rickettsia typhi, Rickettsia prowazekii, Rickettsia sibirica, Saccharomyces cerevisiae, Saccharopolyspora erythraea, Salmonella enterica, Salmonella typhimurium, Schizosaccharomyces pombe, Shewanella oneidensis, Shigella flexneria, Sinorhizobium meliloti, Staphylococcus aureus, Staphylococcus epidermidis, Streptococcus agalactiae, Streptococcus mutans, Streptococcus pneumonia, Streptococcus pyogenes, Streptococcus thermophiles, Streptomyces avermitilis, Streptomyces coelicolor, Sulfolobus solfataricus, Sulfolobus tokodaii, Synechococcus, Synechocystis, Takifugu rubripes, Tetraodon nigroviridis, Thalassiosira pseudonana, Thermoanaerobacter tengcongensis, Thermoplasma acidophilum, Thermoplasma volcanium, Thermosynechococcus elongates, Thermotagoa maritime, Thermus thermophiles, Treponema denticola, Treponema pallidum, Tropheryma whipplei, Ureaplasma urealyticum, Vibrio cholera, Vibrio parahaemolyticus, Vibrio vulnificus, Wigglesworthia glossinidia, Wolbachia pipientis, Wolinella succinogenes, Xanthomonas axonopodis, Xanthomonas campestris, Xylella fastidiosa, Yarrowia lipolytica, Yersinia pseudotuberculosis and Yersinia pestis.

Furthermore, such Mg transporters can be added to any microbe, since the genetic engineering techniques are well known and thousands of species have been engineered to date. Additionally, thousands of vectors are known and available, either from commercial sources, banks and collections, such as ADDGENE, or from colleagues. Thus, it is within the ordinary skill in the art, e.g., to put any of the above genes into yeast, such as Saccharomcyes and Candida, or any of the yeast species named above. Indeed, yeast with overexpressed TE are already available and have increased fat production (see e.g., Leber 2014).

It is also possible to genetically modify many species of algae, including e.g., Spirulina, Apergillus, Chlamydomonas, Laminaria japonica, Undaria pinnatifida, Porphyra, Eucheuma, Kappaphycus, Gracilaria, Monostroma, Enteromorpha, Arthrospira, Chlorella, Dunaliella, Aphanizomenon, Isochrysis, Pavlova, Phaeodactylum, Ulkenia, Haematococcus, Chaetoceros, Nannochloropsis, Skeletonema, Thalassiosira, and Laminaria japonica, plus any of the algal species named above. Indeed, Blatti (2012) already describes a Chlamydomonas reinhardtii with plant TE added thereinto that showed increased FA production. Further, algae are already scaled up for commercial production levels. The microalga Pavlova lutheri is being used as a source of economically valuable docosahexaenoic (DHA) and eicosapentaenoic acids (EPA), and Crypthecodinium cohnii is the heterotrophic algal species that is currently used to produce the DHA used in many infant formulas.

Bacteria from a wide range of species have been successfully modified, and may be the easiest to transform and culture, since the methods were invented in the 70's and are now so common place, that even school children perform genetic engineering experiments using bacteria. Such species include e.g., Bacillus, Streptomyces, Azotobacter, Trichoderma, Rhizobium, Pseudomonas, Micrococcus, Nitrobacter, Proteus, Lactobacillus, Pediococcus, Lactococcus, Salmonella, and Streptococcus, or any of the sequenced bacterial species named above.

Thus, although data is provided in E. coli using various TE genes, various vectors, and various genetic backgrounds, the methods can be easily applied to any species that can be genetically engineered and grown in culture. Indeed, besides bacteria, fatty acid overproduction has been established in cyanobacteria and yeast. Exemplary species include at least the following:

| Species | Genotype | Acc. no and/or vector |
|---|---|---|
| B. subtilis | ΔfadD ΔpfkA short TE⁺, | CAA99571.1, |
| | long TE⁺ MgtA (NP_313246) | YP_007534906.1, pXZcp88, |
| | | pDHC29 vector |
| S. enterica | ΔpfkA short TE⁺, long TE⁺ | EDZ28622.1, pXZcp88, |
| | MgtA (NP_313246) | pDHC29 vector |
| S. aureus | ΔfadD ΔpfkA short TE⁺, | YP_492941.1, |
| | long TE⁺ MgtA (NP_313246) | NP_374809.1, |
| | | pXZcp88, pDHC29 vector |
| S. pichia | short TE⁺ MgtA | AAC49180 P43553 |
| | | pIC vectors or pAO815 |

The method of growing the microbes can be any known in the art. Although some experiments herein use batch culture or fed batch culture, continuous fermentations offer an even higher potential, because the cells can be kept under optimal conditions and in the most suitable growth phase. Other culture methods may be suitable for yeast and/or algae, especially algae, which require light and whose optimal culture methods are still being developed.

Since the product must be somehow purified, the microbial medium can be periodically or continually harvested for free fatty acids and/or derivatives thereof. Lui, for example, used a fed-batch fermentation system, and at 10 hours after induction, the culture medium was pumped through a tributylphosphate phase at a rate of 0.8% per minute (volume for extraction per volume cultivation medium). After passage of the tributylphosphate phase, the culture medium was pumped back into the fermenter vessel. By this process, a total fatty acid production of roughly 9 g/L was achieved [Liu, 2012].

We contemplate the production of FFA herein, but also the production of FFA derivatives, such as free fatty acid esters (FFAE), triacylglycerols (TAG), and the like. Thus, additional genes can be added for these final conversions. These molecules (FFAE, TAG) are considered “derivatives” of free fatty acids herein. Additional derivatives might include hydroxyfatty acids, dicarboxylic acids, aminofatty acids, odd chain fatty acids and unsaturated fatty acids.

The invention can comprise any of the following embodiments, in any combination thereof:
A method of producing fatty acids (or a derivative thereof), comprising: culturing a microbe in a culture medium under conditions effective for the production of fatty acids, wherein said culture medium is supplemented with Mg, and harvesting said fatty acids (or a derivative thereof) from the microbe or the culture medium or both, wherein more fatty acids are produced in said method than a comparable method without Mg supplementation.
A method of producing fatty acids (or a derivative thereof), comprising: culturing a microbe having an overexpressed magnesium transporter gene in a culture medium under conditions effective for the production of fatty acids, wherein said culture medium is optionally supplemented with Mg, and harvesting said fatty acids (or a derivative thereof) from the microbe or the culture medium or both, wherein more fatty acids are produced in said method than a comparable microbe without said overexpressed magnesium transporter gene. Preferably, the organism has an overexpressed TE gene, preferably one described herein. Also, preferred, the medium is supplemented with Mg.
A method of producing fatty acids, comprising: culturing E. coli in a culture medium under conditions effective for the production of fatty acids, wherein said culture medium is supplemented with magnesium, preferably MgCO₃; and harvesting said fatty acids (or a derivative thereof) from the E. coli or the culture medium or both; wherein more fatty acids are produced in said method than a comparable method without said supplementation. Preferably, the organism has an overexpressed TE gene, preferably one described herein.
An improved method of biological production of fatty acid, the method requiring culturing a microbe in a culture medium for a time sufficient to produce fatty acid and harvesting said fatty acid (or a derivative thereof), the improvement comprising i) supplanting said medium with Mg, or ii) adding an overexpressed magnesium transporter gene to said microbe or iii) both i) and ii). Preferably, the organism has an overexpressed TE gene, preferably one described herein.
A microbe comprising an overexpressed magnesium transporter gene and an overexpressed ACP thioesterase (TE) gene, preferably the microbe also having ΔfadD; ΔfadD ΔptsG; ΔfadD ΔpfkA; ΔfadD ΔptsG ΔfadD ΔpfkA; or ΔfadD ΔfadR ΔsucC and overexpression of fabZ⁺ or any of the additional mutations described anywhere herein in any combination.
Any method as described herein wherein Mg is supplemented at a level of >20 mM, >50 mM, >100 mM, or >200 mM.
Any method as described herein wherein fatty acid production is increased at least 2 fold, 10 fold, 20 fold, or 40 fold.
Any method wherein said microbe is a genetically engineered E. coli as described herein, or any microbe as described in the literature cited herein.

E. coli gene and protein names (where they have been assigned) can be ascertained through ecoliwiki.net/ and enzymes can be searched through brenda-enzymes.info/.ecoliwiki.net/ in particular provides a list of alternate nomenclature for each enzyme/gene. Many similar databases are available including UNIPROTKB, PROSITE; 5 EC2PDB; ExplorEnz; PRIAM; KEGG Ligand; IUBMB Enzyme Nomenclature; IntEnz; MEDLINE; and MetaCyc, to name a few.

We have typically used the gene and protein names from BRENDA herein, but not always. By convention, genes are written in italic, and corresponding proteins in regular font. E.g., fadD is the gene encoding FadD or acyl-CoA synthetase.

Generally speaking, we use the gene name and protein names interchangeably herein, based on the protein name as provided in BRENDA. The use of a protein name as an overexpressed protein (e.g., FabH⁺) signifies that protein activity levels can be modulated in ways other than by adding a vector encoding same, since the protein can be upregulated in other ways (e.g., by adding activators or removing inhibitors, etc.). The use of FadD⁻ signifies that the protein can be downregulated in similar way, whereas the use of ΔfadD means that the gene has been directly downregulated, e.g., by knockout or null mutation.

As used herein, “enhanced amount” means at least a 50% improvement in yield of fatty acids comparing to yield of fatty in a control experiment. Preferably, the fatty acid levels increased 1 fold, 1.5 fold, 2 fold, 3, fold, 5 fold or even 10, 20, 30 or 40 fold improvement. Here the calculation of yield is determined by the ratio of grams of fatty acids produced to grams of glucose used. Preferably, at least 20, 25, 30, 35, 40, 45, 50, 60, 70 or 80% improvement is observed.

As used herein, “fatty acids” means any saturated or unsaturated aliphatic acids having the common formula of CₙH_{2n±x}COOH, wherein x≦n, which contains a single carboxyl group.

As used herein, “mutated gene” means a gene that has been engineered to show overexpressed or reduced expression of the gene, as measured by a corresponding change in protein activity. The method of engineering the mutation is not limited, as long as the mutated phenotype is observed.

As used herein, “reduced activity” or “inactivation” is defined herein to be at least a 75% reduction in protein activity, as compared with an appropriate control species. Preferably, at least 80, 85, 90, 95% reduction in activity is attained, and in the most preferred embodiment, the activity is eliminated (100%). Proteins can be inactivated with inhibitors, by mutation, or by suppression of expression or translation, by knock-out, by adding stop codons, by frame shift mutation, and the like. By “null mutant” or “null mutation” what is meant is that the mutation produces undetectable active protein. A gene can be completely (100%) reduced by knockout or removal of part of all of the gene sequence. Use of a frame shift mutation, early stop codon, point mutations of critical residues, or deletions or insertions, and the like, can also completely inactivate (100%) gene product by completely preventing transcription and/or translation of active protein. All null mutants herein are signified by Δ.

As used herein, “overexpression” or “overexpressed” is defined herein to be at least 150% of protein activity as compared with an appropriate control species. Preferably, the activity is increased 200-500%. Overexpression can be achieved by mutating the protein to produce a more active form or a form that is resistant to inhibition, by removing inhibitors, or adding activators, and the like. Overexpression can also be achieved by removing repressors, adding multiple copies of the gene to the cell, or up-regulating the endogenous gene, and the like. All overexpressed genes or proteins are signified herein by “^{k}”.

As used herein, the expressions “microbe,” “strain” and the like may be used interchangeably and all such designations include progeny. It is also understood that all progeny may not be precisely identical in DNA content, due to deliberate or inadvertent mutations. Mutant progeny that have the same function or biological activity as screened for in the originally transformed cell are included. Where distinct designations are intended, it will be clear from the context.

“Operably associated” or “operably linked”, as used herein, refer to functionally coupled nucleic acid or amino acid sequences.

“Recombinant” is relating to, derived from, or containing genetically engineered material. In other words, the genome was intentionally manipulated in some way.

The use of the word “a” or “an” when used in conjunction with the term “comprising” in the claims or the specification means one or more than one, unless the context dictates otherwise.

The term “about” means the stated value plus or minus the margin of error of measurement or plus or minus 10% if no method of measurement is indicated.

The use of the term “or” in the claims is used to mean “and/or” unless explicitly indicated to refer to alternatives only or if the alternatives are mutually exclusive.

The terms “comprise”, “have”, “include” and “contain” (and their variants) are open-ended linking verbs and allow the addition of other elements when used in a claim.

The phrase “consisting of” is closed, and excludes all additional elements.

The phrase “consisting essentially of” excludes additional material elements, but allows the inclusions of non-material elements that do not substantially change the nature of the invention.

The following abbreviations may be used herein:

| Abbrev | Name | Exemplary Acc. Nos. |
|---|---|---|
| ACC | acetyl-CoA carboxylase | NP_414727 P24182.2 |
| AckA | Acetate kinase, in an operon with pta in some species and | P0A6A3 |
| | often both deleted (EC 2.7.2.1) | |
| adhE | Aldehyde-alcohol dehydrogenase, also Alcohol | P0A9Q7 |
| | dehydrogenase (ADH) (EC 1.1.1.1) | |
| ACP or | Acyl-acyl carrier protein | AAB27925.2 |
| acyl-ACP | | |
| Aldolase | aldolase | YP_490034.1 |
| Enolase | enolase | YP_490987.1 |
| FA | Fatty acid | NA |
| FabA | beta-hydroxydecanoyl thioester dehydrase EC: 5.3.3.14 | ABJ00363.1 P0A6Q3 |
| FabB | Component of β-ketoacyl-ACP synthase I (EC 2.3.1.41) | EHY18746.1 P0A953 |
| FabD | malonyl coenzyme A-acyl carrier protein transacylase | AAA23742.1 YP_489360.1 |
| FabF | 3-oxoacyl-[acyl-carrier-protein] synthase 2 (EC 2.3.1.179) | P0AAI5 P0AAI6 |
| FabH | component of β-ketoacyl-acyl carrier protein synthase III | EGT67886.1 P0A6R0 |
| | EC: 2.3.1.180 | |
| FabG | 3-OXOACYL-ACP-beta-Ketoacyl-ACP reductase aka 3- | P0AEK2 |
| | oxoacyl-[ACP] reductase EC 1.1.1.100 | |
| FadL | long-chain fatty acid outer membrane transporter | P10384 |
| FabR | DNA-binding transcription represser | P0ACU5 |
| FabZ | R)-hydroxymyristol acyl carrier protein dehydratase | AAY89693.1 P0A6Q6 |
| | EC: 4.2.1.59 | |
| FadD | fatty acyl-CoA synthetase EC: 6.2.1.3 | EHY19478.1 P69451n |
| | | YP_002999557.1 |
| FadE | Acyl-coenzyme A dehydrogenase (ACDH) (EC 1.3.99.—) | Q47146 C8THQ2 |
| FadR | Represser/activator for fatty acid metabolism regulon | CAA30881.1 P0A8V6 |
| FFA | Free FA | NA |
| FFAE | FFA ester | NA |
| FumAC | fumarase A, fumarase C | YP_006173189.1 and |
| | | YP_489874.1 |
| GapA | component of glyceraldehyde 3-phosphate dehydrogenase-A | YP_490040.1 |
| | complex | |
| GAPDH | Glyceraldehyde-3-phosphate dehydrogenase | AAA23847.1 |
| Glk | glucokinase | EDV65543.1 |
| GltA | citrate synthase | YP_006128080.1 |
| Glucose | glucose phosphotransferase system | YP_490652.1 |
| PTS | | |
| IPTG | Isopropyl β-d-1-thiogalactopyranoside | NA |
| LB | Luria-Bertoni | NA |
| LdhA | Lactate dehydrogenase (EC: 1.1.1.28) | NP_415898 D5D2D6 |
| Medium/ | Acyl-ACP Thioesterase with preference for long chain | ABV54795.1 |
| long TE | FAs ≧C12) | (Ricinus communis) |
| | | ABU96744.1 |
| | | (Jatropha curcas) |
| | | AAX51636.1 |
| | | (Diploknema butyracea) |
| | | AAF02215.1 or |
| | | AF076535.1 |
| | | (Gossypium hirsutum) |
| | | AAB71730 |
| | | (Myristica fragrans) |
| MarR | Multiple antibiotic resistance protein MarR, aka marR, cfxB, | P27245 |
| | inaR, soxQ, b1530, JW5248 | |
| short TE | Acyl-ACP Thioesterase with preference for shorter chain FAs | AAC49180.1 |
| | (C6-<C12) | (Cuphea palustris) |
| | | CAA54060 |
| | | (Cuphea lanceolata) |
| | | AAC72882 |
| | | (Cuphea hookeriana) |
| | | CAC1993 |
| | | (Cuphea lanceolata) |
| | | CAC1993 |
| | | (Cuphea lanceolata) |
| | | AAC49269.1 |
| | | (Cuphea hookeriana) |
| | | AAC49179.1 |
| | | (Cuphea palustris) |
| | | AAC49001 |
| | | (Umbellularia californica) |
| NADK | NAD Kinase, aka yfjB | AAC75664.1 |
| PfkA | phosphofructokinase A | AAC76898.1 CAA26356 |
| PfkB | phosphofructokinase B | AAC74793.1 |
| PflB | Formate acetyltransferase 1 aka Pyruvate formate-lyase 1 | P09373-1 |
| | (EC: 2.3.1.54) | |
| Pgi | phosphoglucose isomerase | AAC76995.1 |
| PGK | phosphoglycerate kinase | YP_491126.1 |
| PGM | phosphoglycerate mutase | AAC75963.1 |
| PK | pyruvate kinase | AAB47952.1 |
| pntAB | Gene encoding pntA NAD(P) transhydrogenase subunit alpha | P07001, BAA15342 |
| | (EC: 1.6.1.2) and PntB-NAD(P) transhydrogenase subunit | P0AB67, YP_489865.1 |
| | beta (EC 1.6.1.2) | |
| pta | Phosphate acetyltransferase (EC: 2.7.2.1) | P0A9M8 |
| PtsG | glucose phosphotransferase enzyme IIBC aka glucose | EHY19964.1 P69786 |
| | permease EC: 2.7.1.69 | |
| pykF | Gene encoding a component of pyruvate kinase I | YP_489938.1 |
| SucC | succinyl-CoA synthetase subunit beta EC: 6.2.1.5 | EFF01582.1 P0A836 |
| TAG | Triacylglycerides | NA |
| TE | acyl ACP thioesterase (TE) | See throughout. |
| TE_{hyb} | Hybrid TE from Ricinus and Cuphea | As described in |
| | | US20140093921 |
| TE_{Rc} | Thioesterase from Ricinus communis | XP_002515564.1 |
| | | XM002515518 |
| TpiA | triose phosphate isomerase | AAC76901.1 |
| UdhA | transhydrogenases | NP_418397.2 |
| PntAB | | YP_489865.1 |
| | | BAA15342.1 |
| udhA | Gene encoding transhydrogenase (UDH), aka sthA | CAA46822 P27306 |
| | (EC: 1.6.1.1) | NP_418397.2, see also |
| | | Q8ZA97 (Shigella); |
| | | Q57H91 (Salmonella); |
| | | Q66G61 (Yersinia), |
| | | D5CGP9 (Enterobacter) |
| | | among thousands of |
| | | available species |

### BRIEF DESCRIPTION OF THE DRAWINGS

FIG. 1. Proposed metabolic map in which the introduction of additional appropriate pathways, the fatty acids can be converted to chemicals.

FIG. 2. Simplified metabolic map showing the fatty acid synthesis pathway. The transcription factor FabR has shown to have negative effect on FabA and FabB; but the transcription factor FadR has the opposite effect on FabA and FabB (Fujita et al., 2007). Free fatty acids are formed in the presence of an acyl-ACP thioesterase, which breaks the fatty acid elongation cycle.

FIG. 3A-B. TE proteins characterized by Jing.

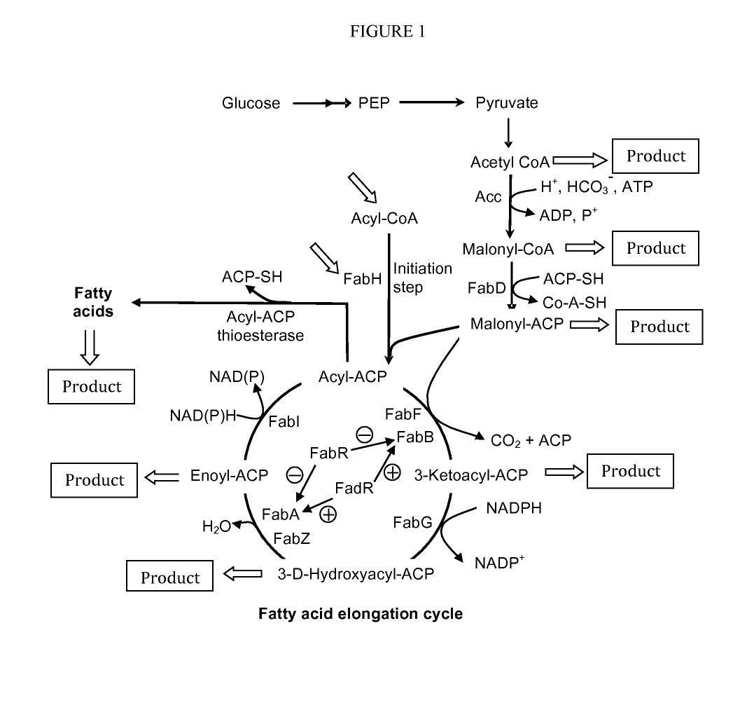

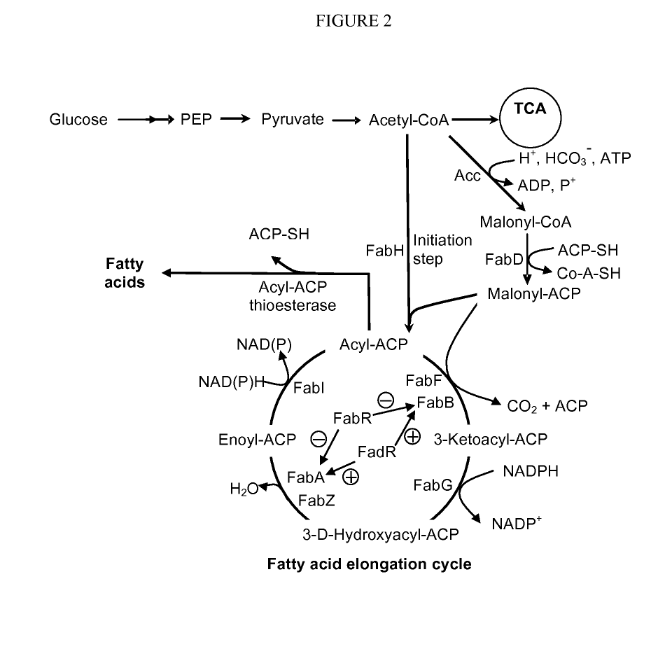

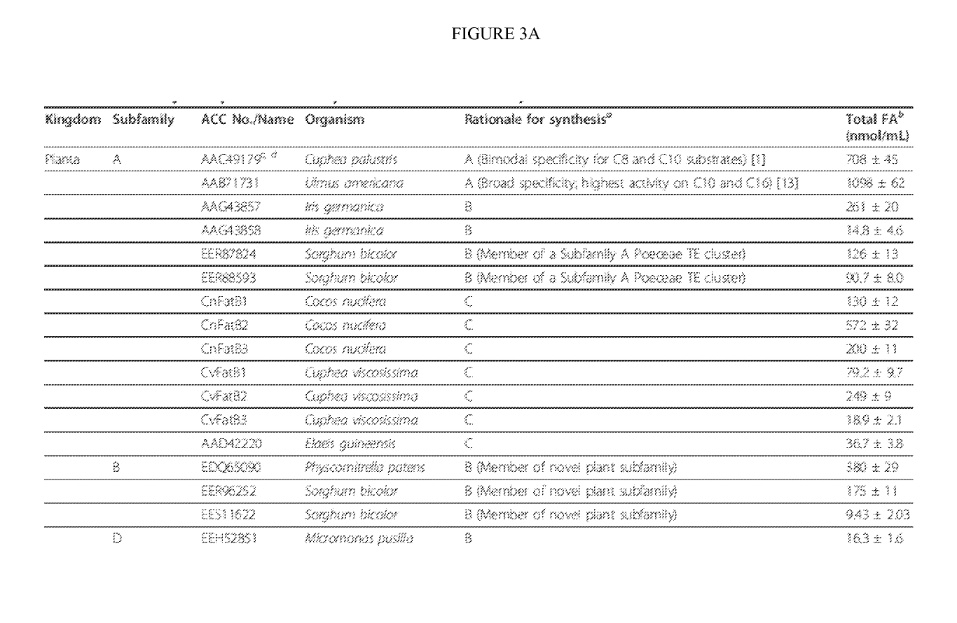

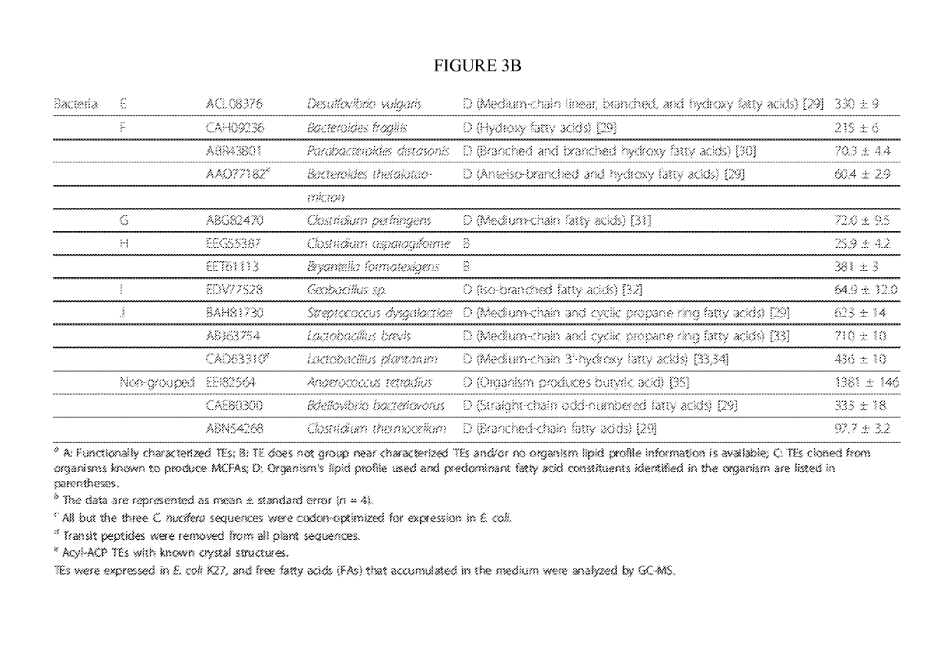

### DETAILED DESCRIPTION

The following experimental descriptions are exemplary only and should not be used to unduly limit the scope of the appended claims and their equivalents. They, plus the abstract, background, summary, figures, tables and the like, are intended however to provide written support for the invention in all its embodiments, whether in graphic, numerical or written form.

### Mg in Medium

To demonstrate improved FA production with increased Mg⁺⁺ in the medium, we used a set of previously engineered high free fatty acid producing E. coli strains ML103 (pTUM3), ML190 (pTUM3), ML191 (pTUM3), ML103 (pXZcp88), ML190 (pXZcp88) and ML191 (pXZcp88) for octanoic acid production and ML212 (pXZ18Z) for medium chain fatty acid production with repeated addition of glucose to increase the titer. However, these strains are exemplary only, and any strain could be used in the culture methods of the invention. The following strains produced fatty acid with carbon chain length ranging from C8 to C16.

The relevant genotype of the strains used is given in Table 1 below.

TABLE 1
Relevant genotype of E. coli strains.

| Strain name | Relevant genotype |
|---|---|
| ML103 (pTUM3) | ΔfadD + an acyl-ACP thioesterase (TE⁺) |
| | from Cuphea hookeriana |
| ML190 (pTUM3) | ΔfadD ΔptsG + TE⁺ from Cuphea hookeriana |
| ML191 (pTUM3) | ΔfadD ΔpfkA + TE⁺ from Cuphea hookeriana |
| ML103 (pXZcp88) | ΔfadD + TE⁺ from Cuphea palustris |
| ML190 (pXZcp88) | ΔfadD ΔptsG + a modified TE⁺ from |
| | Cuphea palustris (as described in |
| | US20140093921-target/leading sequence from |
| | R. communis and remainder from Cuphea palustris)) |
| ML191 (pXZcp88) | ΔfadD ΔpfkA + a modified TE⁺ from |
| | Cuphea palustris |
| ML212 (pXZ18Z) | ΔfadD ΔfadR ΔsucC + overexpression of |
| | fabZ + TE⁺ from Ricinus communis |

Aerobic shake flasks experiments were performed at 30° C. with shaking at 250 rpm for 48 hours with 1% inoculation in 50 ml LB broth medium supplied with 15 g/l glucose and appropriate quantities of ampicillin. An appropriate amount of CaCO₃ and MgCO₃ were also added to the medium. Samples were taken at 24 and 48 hours. Results in Table 2 are averages of triplicate experiments.

TABLE 2
Percentage improvement of octanoic acid production

| | | MgCO₃ ~150 mm | | CaCO₃ ~150 mm | |
|---|---|---|---|---|---|
| | Control | | % im- | | % im- |
| | Octanoic | Octanoic | prove- | Octanoic | prove- |
| Strain name | acid (g/L) | acid (g/L) | ment | acid (g/L) | ment |
| 24 h | | | | | |
| ML103::TUM3 | 0.038 | 0.671 | 1677.808 | 0.148 | 291.160 |
| ML190::TUM3 | 0.732 | 1.432 | 95.601 | 0.590 | −19.382 |
| ML191::TUM3 | 0.577 | 1.009 | 74.776 | 0.577 | 0.008 |
| 48 h | | | | | |
| ML103::TUM3 | 0.039 | 1.632 | 4075.776 | 0.180 | 360.319 |
| ML190::TUM3 | 1.561 | 2.065 | 32.267 | 0.991 | −36.509 |
| ML191::TUM3 | 1.051 | 1.967 | 87.165 | 0.992 | −5.562 |

ML103::TUM3 = ΔfadD short chain acyl-ACP thioesterase⁺ under a constitutive promoter
ML190::TUM3 = ΔfadD ΔptsG short chain acyl-ACP thioesterase⁺ under a constitutive promoter
ML191::TUM3 = ΔfadD ΔpfkA short chain acyl-ACP thioesterase⁺ under a constitutive promoter

The improvement of fatty acid production with supplementation of magnesium was demonstrated first using three strains carrying an acyl-ACP thioesterase from Cuphea hookeriana. For the ML103(TUM3) strain, addition of either CaCO₃ or MgCO₃ to the medium drastically increases the production of octanoic acid as the ML103(TUM3) produces negligible quantity of fatty acids of less than 0.04 g/L. At 48 hrs, addition of CaCO₃ and MgCO₃ showed an improvement of more than 360% and 4000%, respectively (see Table 2). Thus, addition of 150 mg MgCO₃ can improve FFA production more than 40 fold!

At 48 hours the two metabolically engineered strains, ML190(TUM3) and ML191(TUM3), produced 1.561 and 1.0511 g/L of fatty acids respectively. These observations are consistent with earlier reports that the engineered strains exhibit better performance than the parent strain ML103(TUM3). Even with this improvement, the addition of MgCO₃ can further improve the fatty acid production to about 2 g/L while the addition of CaCO₃ showed a negative effect (Table 2).

The experiment was then repeated, but with different plasmid vectors encoding a different TE. Aerobic shake flasks experiments were performed at 30° C. with shaking at 250 rpm for 48 hours with 1% inoculation in 50 ml LB broth medium supplied with 15 g/l glucose and appropriate quantities of ampicillin. The concentration of the inducer, IPTG, is 1 mM. An appropriate amount of CaCO₃ and MgCO₃ were also added to the medium. Samples were taken at 24 hours and 48 hours. Results shown in Table 3 are the averages of triplicate experiments.

TABLE 3
Percentage improvement of octanoic acid (C8) production.

| | | MgCO₃ ~150 mm | | CaCO₃ ~150 mm | |
|---|---|---|---|---|---|
| | Control | | % im- | | % im- |
| | Octanoic | Octanoic | prove- | Octanoic | prove- |
| Strain name | acid (g/L) | acid (g/L) | ment | acid (g/L) | ment |
| 24 h | | | | | |
| ML103::pXZcp88 | 0.274 | 0.674 | 145.609 | 0.130 | −52.685 |
| ML190::pXZcp88 | 0.624 | 1.192 | 90.961 | 0.398 | −36.229 |
| ML191::pXZcp88 | 0.570 | 1.198 | 110.072 | 0.272 | −52.223 |
| 48 h | | | | | |
| ML103::pXZcp88 | 0.267 | 1.172 | 339.542 | 0.135 | −49.491 |
| ML190::pXZcp88 | 1.211 | 1.962 | 62.028 | 0.596 | −50.786 |
| ML191::pXZcp88 | 0.901 | 1.666 | 84.874 | 0.464 | −48.472 |

ML103::pXZcp88 = ΔfadD short chain acyl-ACP thioesterase⁺ under an inducible promoter
ML190::pXZcp88 = ΔfadD ΔptsG short chain acyl-ACP thioesterase⁺ under an inducible promoter
ML191::pXZcp88 = ΔfadD ΔpfkA short chain acyl-ACP thioesterase⁺ under an inducible promoter

Thus, Table 3 shows similar results were obtained with the same host strains but with a different plasmid construct, pXZcp88, which contained a modified TE+ from Cuphea palustris.

The addition of MgCO₃ further improved the fatty acid production while the addition of CaCO₃ showed a negative effect (Table 3). Thus, the method is generally applicable to a variety of TE gene and/or vectors.

A final experiment combined magnesium supplementation with batch feeing of glucose, to further improve yields. Aerobic shake flasks experiments were performed at 30° C. with shaking at 250 rpm for 48 hours with 1% inoculation in 50 ml LB broth medium supplied with 15 g/l glucose and appropriate quantities of ampicillin. The concentration of the inducer, IPTG, is at 1 mM. Two additional batches of glucose of were added to the culture at appropriate times. In addition, an appropriate amount of MgCO₃ was added after the first batch. Results shown in Table 4 are averages of triplicate experiments at the end of second addition of glucose.

TABLE 4
Repeated feed experiment.

| | Fatty | | Overall yield | |
|---|---|---|---|---|
| | acid | % | (g fatty acids/ | % |
| Conditions | (g/L) | improvement | g glucose) | improvement |
| ML212 (pXZ18Z) | 7.529 | — | 0.2064 | — |
| ML212 (pXZ18Z) | 9.629 | 27.89 | 0.2103 | 1.91 |
| with MgCO₃ | | | | |

ML212 (pXZ18Z) = ΔfadD ΔfadR ΔsucC + overexpression of fabZ and a medium chain acyl-ACP thioesterase⁺ under an inducible promoter system

This example involved the repeated addition of glucose in order to increase the final fatty acid titer. The addition of MgCO₃ leads to an improvement of more than 27% in fatty acid production and a small improvement in fatty acid/glucose yield (Table 4).

### Mg Transporter

Studies have revealed that higher concentration of short chain length fatty acids larger than 40 mM have an inhibitory effect on the growth of E. coli, indicating the toxicity of short chain length fatty acids [Royce 2013]. This toxic effect of short chain fatty acids is thought to the result of disrupted membrane integrity, explained by altered membrane fluidity and also increased leakage through membrane pores. Membrane leakage is mainly measured by the loss of Mg⁺⁺ from intracellular region resulting from the toxic effect of desired products. Indeed, challenge with 30 mM octanoic acid resulted in the release of 46% more magnesium than the control sample (Royce, 2013).

Considering the global requirement of Mg⁺⁺ as a cofactor, the loss of Mg⁺⁺ creates severe problems for bacterial growth and maintenance [Smith, 1998], as does the disruption of the membrane for any membrane associated pathways, such as the electron transfer pathway. To prove the necessity of Mg⁺⁺, the growth of mutants having inactivation of all three magnesium transporter encoding genes has been measured, and showed that supplementation of magnesium is required to complement the absence of the three magnesium transporters [Hmiel, 1994]. Without such supplementation, the cells cannot grow.

Thus, methods of addressing fatty acid toxicity via magnesium loss need to be addressed in order to mitigate fatty acid toxicity and further improve FFA production. One method is described above and requires media supplementation with magnesium. However, another possibility is to replace or augment the magnesium transporter activity. This can be combined either with or instead of media supplementation with Mg⁺⁺.

A wide range of magnesium transporters have been identified and characterized in various bacteria, including Salmonella and E. coli [Moncrief, 1999]. Three magnesium transporters have been reported in Salmonella typhimurium. CorA was found to be serving as dominant magnesium transporter that is consistently expressed. CorA mediates both influx and efflux of Mg⁺⁺ along with other divalent ions such as Mn⁺⁺, Co⁺⁺, and so on with different kinetics. In addition to this primary magnesium transporter, in E. coli ATP-dependent magnesium transporters, including MgtA and MgtB, were found to be expressed in response to lower intracellular concentrations of Mg⁺⁺, which shuts down CorA expression. MgtA and MgtB are under the regulation of PhoPQ system.

The selectivity of the Mgt-like transporters is greater than CorA-like transporters, although their kinetic parameters appear to be similar [Park 1976]. This suggests that the Mgt gene families are more likely to mediate Mg⁺⁺ transport under conditions where bacterial cells keep losing intercellular molecules due to damaged membranes caused by high free fatty acid levels.

As discussed above, our group observed a dramatic increase in the titer of octanoic acid when short chain free fatty acid producing E. coli strains were supplemented with Mg⁺⁺ However, to be industrially feasible, an endogenous way to supply Mg⁺⁺ rather than exogeneous supply would be beneficial. As a way of improving the activity of Mg⁺⁺ uptake, we have overexpressed various magnesium transporters in those E. coli that accumulate free fatty acids with carbon length C8 constitutively.

As one example, we have constructed a vector carrying two genes that encode i) a MgtA magnesium transport under the control of inducible promoter and ii) a short chain acyl-ACP thioesterase under the regulation of a constitutive promoter, respectively.

In our preliminary data, Table 5 shows the accumulation of octanoic acid as a unit of titer in one engineered E. coli strain when Mg⁺⁺ is supplemented either endogeneously (e.g., with overexpressed magnesium transporter gene) versus exogenously (e.g., just added in excess to medium). Compared to controls intended to produce octanoic acid without any genetic or environmental perturbations due to constitutive expression of the TE, MgtA carrying strains had a significant impact on the accumulation of octanoic acid, showing 25% improvement even if there is no induction, indicating cis-regulatory effect between genes.

TABLE 5
Accumulated octanoic acid quantity in the presence of Mg²⁺ endogeneously and exogeneously.
Octanoic acid (g/L) with no inducer

| | | Control | MgtA+* | | MgCO₃** | | CaCO₃ | |
|---|---|---|---|---|---|---|---|---|
| | | Octanoic | Octanoic | | Octanoic | | Octanoic | |
| Strain | | acid | acid | | acid | | acid | |
| name | Time | (g/L) | (g/L) | % improvement | (g/L) | % improvement | (g/L) | % improvement |
| ML190::TUM3 | 24 h | 0.732 | 1.039 | 41.970 | 1.432 | 95.601 | 0.590 | −19.382 |
| | 48 h | 1.561 | 1.956 | 25.334 | 2.065 | 32.267 | 0.991 | −36.509 |

ML190—ΔfadD ΔptsG
TUM3—short chain acyl-ACP specific thioesterase under the control of an inducible promoter
MgtA+—E. coli magnesium transporter under control a constitutive promoter (aka Mg²⁺/Ni²⁺ ATPase transporter)
*growth in LB which typically has only less than 0.25 mM magnesium
**LB broth plus 10 g/l MgCO₂ 10 g/l × 1/68 g/m = 0.147 mol/l ~150 mm

Thus, we anticipate that even higher levels will occur when the gene is fully induced, and, further, when combined with excess magnesium levels in the medium. Further improvements will be made by combining these features with the various microbes already developed or future microbes that have increase FFA production.

### Fabz+, TE+, Mtg+

Next we sought to test a strain with just the transporter, but without extra Mg⁺⁺ to show improvement in FFA levels even without high magnesium supplementation. Furthermore, we chose a medium chain TE, instead of the short chain (C8) TE exemplified above.

The strain ML103 has ΔfadD ΔfadR ΔsucC and two plasmids were added to it. One plasmid is the pWL1TZ which carries the fabZ gene from E. coli (FabZ⁺) and a medium chain acyl-ACP thioesterase (TO under a constitutive promoter system. A second compatible plasmid pBAD-mtgA carries the E. coli mtgA gene (MtgA⁺) under an inducible promoter system. FabZ was added to increase the fatty acid titer, as we have previously shown. The function of MgtA does not depend of FabZ.

Aerobic shake flasks experiments were performed at 30° C. with shaking at 250 rpm for 48 hours with 1% inoculation in 50 ml LB broth medium supplied with 15 g/1 glucose and appropriate quantities of ampicillin and chloramphenicol. The concentration of the inducer, IPTG, is at 5 μM to induce the expression the magnesium transporter protein MtgA. LB broth has very small amounts of Mg⁺⁺ that is present in the tryptone and/or yeast extract, about 5-250 μM, but no other magnesium was added. Results shown in Table 6 are averages of triplicate experiments at the end of first and second addition of glucose.

TABLE 6
Repeated feed experiment.

| | | | Overall | |
|---|---|---|---|---|
| | | | yield | |
| | Fatty | | (g fatty | |
| | acid | % | acids/g | % |
| Conditions | (g/L) | improvement | glucose) | improvement |
| First batch feed | | | | |
| ML103(pWL1TZ + | 2.54 | — | 0.184 | — |
| pBAD33) | | | | |
| ML103(pWL1TZ + pBAD- | 2.76 | 8.7 | 0.170 | 8.2 |
| mgtA) | | | | |
| End of third batch feed | | | | |
| ML103(pWL1TZ + | 5.73 | — | 0.128 | — |
| pBAD33) | | | | |
| ML103(pWL1TZ + pBAD- | 6.21 | 8.4 | 0.138 | 8.7 |
| mgtA) | | | | |

ML103(pWL1TZ + pBAD33) = ΔfadD ΔfadR ΔsucC + overexpression of fabZ and a medium chain acyl-ACP thioesterase⁺ under a constitutive promoter system
ML103(pWL1TZ + pBAD33-mtgA) = ΔfadD ΔfadR ΔsucC + overexpression of fabZ and a medium chain TE⁺ under a constitutive promoter system and mgtA under an inducible promoter system

Although this level of FFA improvement was modest (<10%), the FFA levels can be further improved by improving the culture conditions. Thus, the same bacteria were tested in a bioreactor, as opposed to shaker flask experiments. Bioreactors can lead to increased yields because it is easier to control temperature, dissolved oxygen levels and pH and other parameters such as nutrient feeding rate during a fed-batch operating mode.

The bioreactor experiments were performed with two strains. Strain MG105(pWL1TZ, pBAD33), which carries two compatible plasmids with one plasmid contains an acyl-ACP thioesterase and the E. coli fabZ gene together as an operon and another plasmid as the control. Strain MG105(pWL1TZ, pBAD33-mtgA), which carries two compatible plasmids with one plasmid contains an acyl-ACP thioesterase together as an operon and the E. coli fabZ gene and another plasmid contains the E. coli mtgA gene.

Aerobic experiments were performed at 30° C. in a 1-L bioreactor system with dissolved oxygen controlled at 40% and pH at 7.0. LB broth medium supplied with 15 g/l glucose and appropriate quantities of ampicillin and chloramphenicol was used. The concentration of the inducer, IPTG, was at 5 μM to induce the expression the magnesium transporter protein MtgA. Additional glucose (15 g/L) was added during the experiment at an appropriate time (24 hrs and 48 hrs). Results shown in table 7 are averages of triplicate experiments at the end of the second addition of glucose.

TABLE 7
Repeated feed experiment.

| Conditions | Fatty acid (g/L) | % improvement |
|---|---|---|
| ML103(pWL1TZ + pBAD33) | 4.41 | — |
| ML103(pWL1TZ + pBAD- | 5.88 | 33 |

ML103(pWL1TZ + pBAD33) = ΔfadD ΔfadR ΔsucC + overexpression of fabZ and a medium chain acyl-ACP thioesterase⁺ under a constitutive promoter system
ML103(pWL1TZ + pBAD33-mtgA) = ΔfadD ΔfadR ΔsucC + overexpression of fabZ and a medium chain acyl-ACP thioesterase⁺ under a constitutive promoter system and mgtA under an inducible promoter system

We can see that the use of a bioreactor, as opposed to shaker flasks, improved yield more than 30%. Further improvements are possible by optimizing culture conditions and by increasing magnesium levels in the medium.

In summary, the overexpression of a magnesium transporter also help the production of medium chain fatty acids, even without magnesium supplementation. However, as seen above, supplementing with mm levels of magnesium further improves yields.

The following references are incorporated by reference in their entirety.
Blatti J. L., et al. Manipulating Fatty Acid Biosynthesis in Microalgae for Biofuel through Protein-Protein Interactions. PLoS ONE 7(9): (2012).
Davies, H. M., et al., 1993. Fatty acid synthesis genes: Engineering the production of medium-chain fatty acids. p. 176-181. In: J. Janick and J. E. Simon (eds.), New crops. Wiley, New York.
Fujita Y, et al., Regulation of fatty acid metabolism in bacteria. Mol Microbiol. 2007 November; 66(4):829-39.
Hmiel S P, Snavely M D, Florer J B, Maguire M E, Miller C G: Magnesium transport in Salmonella typhimurium: genetic characterization and cloning of three magnesium transport loci. Journal of bacteriology 1989, 171(9):4742-4751.
JanBen H. J. and Steinbüchel A., Fatty acid synthesis in Escherichia coli and its applications towards the production of fatty acid based biofuels, Biotechnology for Biofuels 2014.
Leber C. & Da Silva N. A., Engineering of Saccharomyces cerevisiae for the synthesis of short chain fatty acids, Biotechnology and Bioengineering 111(2): 347-358 (2014).
Lu, X., et al., 2008. Metabolic Engineering. 10: 333-339.
Liu X, Sheng J, Curtiss R III: Fatty acid production in genetically modified Cyanobacteria. Proc Natl Acad Sci USA 2011, 108:6899-6904.
Liu H, et al., Production of extracellular fatty acid using engineered Escherichia coli, Microb Cell Fact 2012, 11:41.
Moncrief M B, Maguire M E: Magnesium transport in prokaryotes. Journal of biological inorganic chemistry, 1999, 4(5):523-527.
Park M H, Wong B B, Lusk J E: Mutants in three genes affecting transport of magnesium in Escherichia coli: genetics and physiology. Journal of bacteriology 1976, 126(3):1096-1103.
Radakovits R., et al., Genetic Engineering of Algae for Enhanced Biofuel Production, Eukaryot Cell. 9(4): 486-501 (2010).
Randor, R. E., et al., The damaging effects of short chain fatty acids on Escherichia coli membranes. Applied microbiology and biotechnology 2013, 97(18):8317-8327.
Ruffing A M, Jones H D: Physiological effects of free fatty acid production in genetically engineered Synechococcus elongatus PCC 7942. Biotechnol Bioeng 2012, 109:2190-2199.
Smith R L, Maguire M E: Microbial magnesium transport: unusual transporters searching for identity. Molecular microbiology 1998, 28(2):217-226.
Tang X, Feng H, Chen W N: Metabolic engineering for enhanced fatty acids synthesis in Saccharomyces cerevisiae, Metab Eng 2013, 16:95-102.
Zhang X, et al. 2011. Construction and characterization of plant acyl-thioesterase genes for free fatty acid production in Escherichia coli. Metabolic Engineering, in press.
WO2011116279 and WO2013059218 by San.
US20140212935 Short chain fatty acids from bacteria
US20140273114 Bacteria and method for synthesizing fatty acids
US20140093921 Bacteria and method for synthesizing fatty acids
US20140193867 Microbial odd chain fatty acids
US20140335578 Integrated biodiesel process
US20130316413 Reverse beta oxidation pathway

1. A microbe comprising an overexpressed magnesium transporter gene and an overexpressed TE gene.

2. The microbe of claim 1, further comprising:
a) ΔfadD ΔptsG;
b) ΔfadD ΔpfkA;
c) ΔfadD ΔptsG ΔfadD ΔpfkA;
d) ΔfadD ΔfadR ΔsucC and overexpression of fabZ⁺;
e) ΔptsG;
f) ΔpfkA;
g) ΔptsG ΔfadD ΔpfkA;
h) ΔfadR ΔsucC and overexpression of fabZ⁺;
i) ΔsucC;
j) ΔsucC, ΔptsG;
k) ΔsucC, ΔptsG and overexpression of fabZ⁺;
l) ΔfadR;
m) ΔfadR, ΔptsG; or
n) ΔfadR, ΔptsG and overexpression of fabZ⁺.

3. The microbe of claim 1, further comprising any of the following mutations in any combination thereof:
ΔfadD, ΔsucC
ΔfadD, ΔfumAC and optional ΔsucC
ΔfadD, ΔgapA and optional ΔsucC
ΔfadD, ΔptsG and optional ΔsucC
ΔfadD, ΔpfkA and optional ΔsucC
ΔfadD, Δglk and optional ΔsucC
TE⁺ and fabD⁺
TE⁺ and udhA⁺
TE⁺ and pntAB⁺
ΔsucC
ΔfumAC and optional ΔsucC
ΔgapA and optional ΔsucC
ΔptsG and optional ΔsucC
ΔpfkA and optional ΔsucC
Δglk and optional ΔsucC
NAD-kinase⁺
acc⁺ and/or fabD⁺ and/or udhA⁺ and/or pntAB⁺ and/or NAD-kinase⁺
ΔadhE
ΔldhA
Δpta, ΔackA, or both ΔackA-pta
ΔpflB
ΔfadD and/or ΔfadE
ΔrelA
fabZ+
fabG+
fabL+
NAD-dependent fabG+.

4. The microbe of claim 2, further comprising any of the following mutations and/or overexpressions in any combination thereof:
ΔfadD, ΔsucC
ΔfadD, ΔfumAC and optional ΔsucC
ΔfadD, ΔgapA and optional ΔsucC
ΔfadD, ΔptsG and optional ΔsucC
ΔfadD, ΔpfkA and optional ΔsucC
ΔfadD, Δglk and optional ΔsucC
TE⁺ and fabD⁺
TE⁺ and udhA⁺
TE⁺ and pntAB⁺
ΔsucC
ΔfumAC and optional ΔsucC
ΔgapA and optional ΔsucC
ΔptsG and optional ΔsucC
ΔpfkA and optional ΔsucC
Δglk and optional ΔsucC
NAD-kinase⁺
acc⁺ and/or fabD⁺ and/or udhA⁺ and/or pntAB⁺ and/or NAD-kinase⁺
ΔadhE
ΔldhA
Δpta, ΔackA, or both ΔackA-pta
ΔpflB
ΔfadD and/or ΔfadE
ΔrelA
fabZ+
fabG+
fabL+
NAD-dependent fabG

5. A method of producing free fatty acids, comprising:
a) culturing a microbe having
i) an overexpressed acyl ACP thioesterase (TE) gene, and optionally
ii) an overexpressed magnesium transporter gene,
in a culture medium under conditions effective for the production of free fatty acids, wherein said culture medium is supplemented with >20 mm magnesium (Mg), and
b) harvesting said free fatty acids or a derivative thereof from the microbe or the culture medium or both, wherein more free fatty acids or a derivative thereof are produced in said method than a comparable method without Mg supplementation.

6. The method of claim 5, wherein Mg is supplemented at a level >30 mM.

7. The method of claim 5, wherein Mg is supplemented at a level >50 mM.

8. The method of claim 5, wherein Mg is supplemented at a level >100 mM.

9. The method of claim 5, wherein Mg is supplemented at a level >200 mM.

10. The method of claim 6, wherein fatty acid production is increased at least 2 fold.

11. The method of claim 7, wherein fatty acid production is increased at least 10 fold.

12. The method of claim 8, wherein fatty acid production is increased at least 20 fold.

13. The method of claim 8, wherein fatty acid production is increased at least 40 fold.

14. A method of producing fatty acids, comprising:
a) culturing a microbe of claim 1 in a culture medium with at least 20 mm magnesium under conditions effective for the production of free fatty acids; and
b) harvesting said free fatty acids from said microbe or the culture medium or both, wherein more free fatty acids are produced in said method than a comparable microbe without said 20 mm magnesium.

15. The method of claim 14, said culture medium comprising at least 50 mm magnesium.

16. The method of claim 14, said culture medium comprising at least 100 mm magnesium.

17. A method of producing fatty acids, comprising:
a) culturing a microbe of claim 1 in a culture medium with at least 20 mm magnesium under conditions effective for the production of free fatty acids; and
b) harvesting said free fatty acids from said microbe or the culture medium or both, wherein more free fatty acids are produced in said method than a comparable microbe without overexpressed magnesium transporter gene.

18. The method of claim 17, said culture medium comprising at least 50 mm magnesium.

19. The method of claim 17, said culture medium comprising at least 100 mm magnesium.
